SEQUENCE LISTING (20 sequences)

SEQ ID NO: 1 - DNA, 22 bp, Artificial sequence
  primer B1-5382INSCI1 F
cacttccattgaaggaagcttc

SEQ ID NO: 2 - DNA, 20 bp, artificial sequence
  primer B1-5382INSCI1 R
tacctttctgtcctggggat

SEQ ID NO: 3 - DNA, 20 bp, artificial sequence
  primer B1-5382INSCI2 F
tgacgtgtctgctccacttc

SEQ ID NO: 4 - DNA, 20 bp, artificial sequence
  primer B1-5382INSCI2 R
acctttctgtcctggggatt

SEQ ID NO: 5 - DNA, 22 bp, artificial sequence
  primer B1-5382INSCK1 F
cacttccattgaaggaagcttc

SEQ ID NO: 6 - DNA, 20 bp, artificial sequence
  primer B1-5382INSCK1 R
caaaggggagtggaatacag

SEQ ID NO: 7 - DNA, 20 bp, artificial
  primer B1-5382INSCK2 F
atatgacgtgtctgctccac

SEQ ID NO: 8 - DNA, 20 bp, artificial sequence
  primer B1-5382INSCK2 R
caaaggggagtggaatacag

SEQ ID NO: 9 - DNA, 20 bp, artificial sequence
  primer B1EX5IK1 F
ctcttaagggcagttgtgag

SEQ ID NO: 10 - DNA, 20 bp, artificial sequence
  primer B1EX5IK1 R
ttcctactgtggttgcttcc

SEQ ID NO: 11 - DNA, 20 bp, artificial sequence
  primer B1EX5IK2 F
atggctcttaagggcagttg

SEQ ID NO: 12 - DNA, 20 bp, artificial sequence
  primer B1EX5IK2 R
tgtggttgcttccaacctag

SEQ ID NO: 13 - DNA, 21 bp, artificial sequence
  primer B1_4154DELAI1 F
caaaggcatctcaggaacatc

SEQ ID NO: 14 - DNA, 21 bp, artificial sequence
  primer B1_4154DELAI1 R
caagcccgttcctctttctca

SEQ ID NO: 15 - DNA, 20 bp, artificial sequence
  primer B1_4154DELAI2 F
ttggctcagggttaccgaag

SEQ ID NO: 16 - DNA, 20 bp, artificial sequence
  primer B1_4154DELAI2 R
aagcccgttcctctttgtca

SEQ ID NO: 17 - DNA, 20 bp, artificial sequence
  primer B1_4154DELAK1 F
ttggctcagggttaccgaag

SEQ ID NO: 18 - DNA, 21 bp, artificial sequence
  primer B1_4154DELAK1 R
gtgctccccaaaagcataaac

SEQ ID NO: 19 - DNA, 22 bp, artificial sequence
  primer B1_4154DELAK2 F
tcctagccctttcacccataca

SEQ ID NO: 20 - DNA, 21 bp, artificial sequence
  primer B1_4154DELAK2 R
gtgctccccaaaagcataaac

(19) United States
(12) Patent Application Publication
(10) Pub. No.: US 2008/0241834 A1
(43) Pub. Date: Oct. 2, 2008

(54) Method for improving neoadjuvant chemotherapy

(76) Inventors: Tomasz Byrski, Szczecin (PL); Jacek Gronwald, Szcaecin (PL); Jan Lubinski, Szczecin (PL); Tomasz Huzarski, Szczecin (PL); Steven Narod, Ontario (CA)

Correspondence Address: COOPER & DUNHAM, LLP, 1185 AVENUE OF THE AMERICAS, NEW YORK, NY, 10036, US

(21) Appl. No.: 11/809,574

(22) Filed: Jun. 1, 2007

(30) Foreign Application Priority Data
Jun. 1, 2006 (PL) P.379 827

(51) Int. Cl. C12Q 1/68; G01N 33/566

(52) U.S. Cl. 435/6; 436/501

(57) ABSTRACT

Disclosed is a method and composition for optimizing the efficiency of breast cancer neoadjuvant chemotherapy, depending on the particular constitutional genotype characteristics of the gene BRCA1 in each patient. Generally, the invention concerns a new method to improve neoadjuvant therapy depending on a particular constitutional genotype. Subject of invention allow to synthesize DNA and identification of germline BRCA1 genetic abnormalities which are correlated with a significantly decreased clinical response to neoadjuvant chemotherapy based on taxane-derived cytostatics in breast cancer patients.

This application claims priority of Polish patent application no. P.379827, filed Jun. 1, 2006.

### BACKGROUND OF THE INVENTION

Constitutional mutations in the gene BRCA1 are the main factor responsible for high risk monogenic predisposition to breast and ovarian cancer (Ford et al. Am J Human Genet 1998; 62:676-89; Narod et al. Am J Hum Genet 1995; 56; 254-64; Narod et al. Am J Hum Genet 1995; 57:957-8). Biological effects of BRCA1 belong to a reduced group of phenomena, where distinct abnormalities of just one protein lead to very critical consequences, almost independently of modifiers and environmental factors as evidenced here by the very high risk of breast and ovarian cancer in BRCA1 mutation carriers throughout different human populations. To date hundreds of constitutional mutations have been described for BRCA1 (BIC database). Some of these are recurrent mutations with founder effect, i.e. show a population-specific profile. For example, and without loss of generality, there are founder mutations characteristic for the Ashkenazi Jewish (Tonin et al. Nat Medicine 1996; 2:1179-83), Finnish (Huusko et al. Am J Hum Genet 1998; 62:1544-8), Danish (Bergthorsson et al. J Med Genet 2001; 38:361-8), Italian (Russo et al. Breast Cancer Res Treat 2007; in press), English (Anglian Breast Cancer Study Group Br J Cancer 2000; 1301-8), Portuguese (Pexoto et al. Fam Cancer 2006; 5:379-87), Indian (Hedau et al. Breast Cancer Res Treat 2004; 88:177-86; Saxena et al. BMC Med Genet 2006; 7:75; Valarmathi et al. Hum Mutat 2004; 23:205), Japanese (Ikeda et al. Int J Cancer 2001; 91:83-8), Turkish (Yazici et al. Br J Cancer 2000; 83:737-42), Pakistani (Rashid et al. Int J Cancer 2006; 119:2832-9), Korean (Seo et al. Hum Mutat 2004; 24:350; Han et al. Clin Genet 2006; 70:496-501), Dutch (Peelen et al. Am J Hum Genet 1997; 60:1041-9; Petrij-Bosch et al. Nat Genet 1997; 17:341-5), Canadians of French origin (Tonin et al. Am J Hum Genet 1998; 63:1341-51) and Canadians of English origin (Risch et al. Am J Hum Genet 2001; 68:700-10) among many others.

Analogous founder mutations can also be found in Slavic populations, as shared by e.g. the Polish (Gorski et al. Am J Hum Genet 2000; 66:1963-8), the Chech (Machackova et al. Cas Lek Cesk 2000; 139:635-7), the Latvian (Csokay et al. Hum Mutat 1999; 14:92), the Belarusian (Oszurek et al. Clin Genet 2001; 60:470-1), or the Russian (Tereschenko et al. Hum Mutat 2002; 19:184). Polish patent application no. P. 335 917 describes founder mutations of the gene BRCA1 characteristic for the Slavic population. Another Polish patent application with no. P. 364 413 shows that ˜90% of all BRCA1 mutations in Poland belong to one out of three common mutations: BRCA1 ex.20 5382 ins C, BRCA1 ex.5 300T→G and BRCA1 ex.11 4153 del A.

In summary, we can conclude that the current state of the art shows a strong correlation between germline mutations in the gene BRCA1 and predisposition to breast and ovarian cancer, whereas the influence of each particular mutation is different in different ethnic groups.

Taxoids are diterpen compounds used in pharmacy mainly as cytostatics, e.g. paclitaxel (Taxol®) and docetaxel (Taxotere®). Paclitaxel is a highly complex molecule with several sites for chiral bindings of carbon atoms. Paclitaxel was identified in 1960 in a research program of the National Institute of Health (NIH) of the USA committed to the identification of active compounds out of 35 000 plant species. An extract from the bark of the Pacific Yew Tree, Taxus brevifolia, showed interesting cytostatic properties. In 1969 the active compound paclitaxel was isolated from the bark extract and in 1971 its chemical structure could be determined (Rowinsky et al. J National Can Inst 82:11247, 1990).

In contrast to other substances as colchicine or vinca alkaloids, whose antitumoral properties rely on their ability to depolymerize the cell microtubules, paclitaxel mode of action prevents the depolymerization of the microtubules. In this way microtubules are stabilized to the extent that cell division is disrupted (Schiff et al. Nature 277:665, 1979). The FDA allowed commercialization of paclitaxel in 1993 for chemotherapy in breast, ovarian, lung and prostate cancer, melanoma and leukaemia. The effectivity against ovarian, breast and lung cancer is around 30%, 50% and 20% respectively (David et al. J Nat Prod 53, 1990).

Docetaxel also induces the assembly of microtubules, thus building a stable configuration during mitosis that prevents cell division (Katzung's Pharmacology, 9th Edition, 2004).

Docetaxel was presented as a new-generation cytostatic, particularly efficient for chemotherapy against breast cancer (Piccart, Anticancer Drugs 4:7-11, 1995). Currently, many other taxanes and their derivatives are known, as well as the extraction methods and the application for therapy against cancer, including those disclosed in:

| | | | | | |
|---|---|---|---|---|---|
| WO94/14787, | US6916942, | US6750246, | US6610860, | US6476242, | US6369244, |
| US6353120, | US6248908, | US6017935, | US5977386, | US5902822, | US5840929, |
| US5773464, | US5773629, | US4814470, | US4857653, | US4876399, | US4942184, |
| US4960790, | US5278324, | US5283253, and | US5352806. | | |

Thus, both taxane-derived chemotherapeutic drugs and their application will be considered as conventional herein.

The current use of taxanes for chemotherapy against cancer is associated with side-effects. Such include neutropenia, alopecia, debilitation, pains in articulations and muscle tissue, skin reactions, anemia, water retention and even damages of the liver and the heart. It is also evidenced that therapeutical efficiency of those cytostatics is highly variable in different cancer patients. As can be deduced from the foregoing state of the art, an objective problem is the lack of a method that could reliably classify patients in groups of responders and non-responders towards cytostatics prior to therapy such that an alternative drug, e.g. a DNA-damaging drug, may be administered to patients deemed less likely to respond to cytostatic therapy, thus improving therapy success and reducing side-effects. A first method to cope with this specific problem is subject of WO2004042080 and WO2005121786. Based on in vitro studies, these applications suggest that breast cancer cell lines with low BRCA1 activity are not responsive to chemotherapy with taxanes, and thus recommend a DNA damaging agent as a chemotherapeutic agent, instead. Under the possible ways to determine whether BRCA1 activity, is reduced, these applications suggest the analysis of mutations in the gene BRCA1 in breast tumor biopsy material.

However, for the use of that method in clinical practice, some problems arise. A major objection is the generalization of the in vitro model to a human subject. The development of the tumor is influenced by many factors, such as permeability to the tumor cells, interstitial hypertension, metabolic degradation, immune response or angiogenesis among others, that greatly diverge between in vivo and in vitro studies and most remarkably the context of metabolites taken to and from the tumor site by blood circulation, e.g. regulator molecules expressed elsewhere, is completely absent in vitro. This divergences often account for a lack of correlation of the effect of anticancer drugs in vivo and vitro (Williams et al. Cancer Res 2000; 60:6045-51; Poondru et al. Invest New Drugs 2002; 20:23-33, McCready et al. J Natl Cancer Inst 1989; 81: 682-7).

### SUMMARY OF THE INVENTION

Disclosed is a method and composition for optimizing the efficiency of breast cancer neoadjuvant chemotherapy, depending on the particular constitutional genotype characteristics of the gene BRCA1 in each patient. Generally, the invention concerns a new method to improve neoadjuvant therapy depending on a particular constitutional genotype. Subject of invention allow to synthesize DNA and identification of germline BRCA1 genetic abnormalities which are correlated with a significantly decreased clinical response to neoadjuvant chemotherapy based on taxane-derived cytostatics in breast cancer patients.

### DETAILED DESCRIPTION

Subject of this invention is a method for predicting response to neoadjuvant chemotherapy in breast cancer patients, who have already developed a tumor, dependent on their constitutional BRCA1 genotype.

Whenever neoadjuvant therapy is advisable, there is an immediate urge from the clinical point of view in determining the most efficient chemotherapy for the patient, since any delay in the application of the correct therapy reduces its chances of success. In this scenario, a method is needed that allows a fast decision-making for the oncologist.

The subject of the present invention is a method for predicting response to antitumoral taxane chemotherapy of a breast cancer patient, depending on his constitutional BRCA1 genotype, characterized by analysis of any genetic material obtained from the patient. In fact, as the method is focused on germline founder mutations, the prediction of the response to a possible future taxane therapy is already possible at a stage where the individual is just identified as predisposed to breast and ovarian cancer in the frame of a standard genetic scan for cancer associated markers, as is often performed e.g. in families with high cancer incidence or for family members of BRCA1 mutation carriers even in absence of family cancer aggregation.

As mutations of the BRCA1 gene it is understood mutations affecting the genetic sequence of the gene BRCA1, as well as flanking mutations in the direct neighbourhood of BRCA1, which are classified in the database of the Breast Cancer Information Core (BIC). The database is available in the internet under http://research.nhgri.nih.gov/bic/. The numeration system of the genetic sequence and the terminology to denominate the mutations used herein comply with the established scientific terminology in this area. As founder mutations, it is understood those among the aforementioned ones, which appear with a characteristically high frequency in specific human populations with a common ethnical origin.

In the context of this invention, the patient is ideally characterized as a patient of known ethnical origin. As a first example, without loss of generality, the patient is of Slavic origin, whereas the main founder mutations of BRCA1 gene observed in that population are 5382insC, 300T→G and 4153delA. As a second example, without loss of generality, the patient is of Ashkenazi Jewish origin, whereas the main founder mutations of BRCA1 gene observed in that population are 185delAG and 5382insC. Exemplarily, a sample founder mutations characterizing different ethnic populations is summarized in table 1.

TABLE 1
Sample of BRCA1 founder mutations

| Founder BRCA1 | |
|---|---|
| mutations | Ethnic populations |
| S1503X | Pakistani |
| R1835X | Pakistani |
| 185insA | Pakistani |
| 185delAG | Ashkenazi Jewish, Hungarian, Indian, Hispanic, |
| | Pakistani |
| 5382insC | Ashkenazi Jewish, Turkish, Slavic, Hungarian |
| 300T→G | Slavic, Hungarian |
| 4153delA | Slavic |
| 3171ins5 (3166ins5) | Scandinavian |
| 2595delA | Scandinavian |
| 1806C→T | Scandinavian |
| 1201del11 | Scandinavian |
| 1135insA | Scandinavian |
| 1675delA | Scandinavian |
| 2804delAA | Dutch |
| Alu-mediated deletions | Dutch |
| ex13, ex22 | |
| 4446C→T | French Canadian |
| 2953del3 + C | French Canadian |
| 3300delA | Thai |
| Asp67Glu | Thai |
| 2156delinsCC | Portuguese |
| 3450del4 | Portuguese |
| 2552delC | Hispanic |
| R1443X | Hispanic |
| S955X | Hispanic |
| IVS5 + 1G > A | Hispanic |
| Tyr978X | Non-Ashkenazi Jews |
| 943ins10 | West-African |
| IVS13 + 1G > A | West-African |

A genetic analysis of BRCA1 germline mutations based on population-specific panels of known founder mutations is particularly favorable, since it allows a highly reliable identification of the most common alterations in BRCA1 with conventional indirect techniques based on DNA or RNA within a question of hours. The mutations may be detected directly or indirectly at DNA, RNA or protein level, but particularly favorable in the context of this invention is the analysis of DNA or RNA for the indirect identification of mutations with one of the following techniques: ASO PCR (allele specific-polymerase chain reaction), SSCP (single-strand conformation polymorphism), ASA (allele specific analysis), RFLP-PCR (restriction fragment length polymorphism—polymerase chain reaction), Taqman RT-PCR (real-time PCR) or microarray technology. Examples of primers that can be used for the amplification of such sequences of the gene BRCA1 in the context of the present invention are presented in tables 2 and 3.

Analogously, it is also favorable the genetic analysis of BRCA1 germline mutations based on a larger, unspecific panel comprising all known BRCA1 founder mutations, or a sample of the most frequent ones, to be applied for patients with unknown ethnic origin.

In the context of this invention, the biological material subject of genetic analysis is not necessarily a tumor biopsy. In the contrary, somatic changes are more difficult to identify and mostly require time-consuming direct DNA or RNA sequencing techniques since, unlike founder mutations, they may occur at any position of the sequence. It is of critical relevance in clinical practice to assign the correct neoadjuvant chemotherapy, whenever needed, as soon as possible. Thus, the identification of the constitutional BRCA1 genotype should be preferably performed on biological material as easily available as possible, such as peripheral blood or saliva. This is a clear advantage in comparison with tumor biopsies, where the access to tumor material is more difficult and sometimes impossible.

In the context of this invention, taxane cytostatic drugs comprise paclitaxel, docetaxel and their known derivates and analogues, treated tumors comprise malignancies occurring with increased probability among BRCA1 mutation carriers such as prostate cancer, leukaemia, lymphoma and particularly breast and ovarian cancer, and mutations of the gene BRCA1 are constitutional mutations.

The invention is described in the following example of application, to better illustrate its relevance. However, the invention cannot be reduced to the mentioned examples.

### EXAMPLE

Reduced response to neoadjuvant taxane therapy in breast cancer patients, which carry a constitutional mutation in the gene BRCA1.

One of the key clinical issues that must be addressed in the treatment of hereditary breast cancer is choice of chemotherapy. Unlike chemotherapy given after surgery, the effects of neoadjuvant chemotherapy can be assessed quickly by measuring tumor size and lymph node status before and after treatment. Although response does not invariably predict a patient's ultimate outcome, rates of complete response correlate well with survival rates (Fisher et al. J Clin Oncol 1998; 16:2672-85).

3479 unselected incident cases of invasive breast cancer were identified at 18 different hospitals in Poland during the study period. The medical records and pathology reports were reviewed locally at the study centre in Szczecin. Information was recorded on age at diagnosis, stage, grade and lymph-node status, estrogen-receptor status, multi-centricity and bilaterality. Pathology review of tumor blocks and/or paraffin-embedded slides were requested from the corresponding pathology centres. One or more specimens were obtained from 3136 of the 3472 patients. A central pathology review was conducted in Szczecin by two pathologists associated with the study. Pathologists were blinded to mutation status. Each case was reviewed with regard to histology (medullary, ductal, lobular, tubulolobular or other). Representative slides were obtained from 66 patients (78%). These were stained for estrogen-receptor, progesterone receptor and ERBB2. Where slides were unavailable, information on estrogen-receptor and progesterone receptor was abstracted from the pathology report.

A mutation analysis of the BRCA1 gene was carried out for mutations 4153delA and 5328insC by a multiplex allele-specific polymerase chain reaction (PCR) assay. A third common mutation (C61G) was detected with the help of a restriction enzyme site in exon 5 specific for that mutation. 3472 of the 3479 patients (99.8%) could be successfully genotyped.

Identification of several mutations may be carried out grouped or independently. In the former case the primers set comprises an oligonucleotide pair for the identification of the mutation and a second oligonucleotide pair for control (table 2). However, it is particularly favourable the use of primer sets for a single multiplex PCR reaction (table 3).

TABLE 2

Primer sets for analysis of BRCA1 mutations with
conventional PCR.

| Primer | | | Primer for sense | Primer for antisense |
|---|---|---|---|---|
| pairs | Primer ID | Function | strand [F] 5′->3′ | strand [R] 5′->3′ |
| Pair I for | B1-5382INSCI1 | identification | CAC TTC CAT TGA | TAC CTT TCT GTC CTG |
| BRCA1 ex.20 | | | AGG AAG CTT C | GGG AT |
| 5382 ins C | | | | |
| Pair 2 for | B1-5382INSCI2 | identification | TGA CGT GTC TGC | ACC TTT CTG TCC TGG |
| BRCA1 ex.20 | | | TCC ACT TC | GGA TT |
| 5382 ins C | | | | |
| Pair 3 for | B1-5382INSCK1 | control | CAC TTC CAT TGA | CAA AGG GGA GTG GAA |
| BRCA1 ex.20 | | | AGG AAG CTT C | TAC AG |
| 5382 ins C | | | | |
| Pair 4 for | B1-5382INSCK2 | control | ATA TGA CGT GTC | CAA AGG GGA GTG GAA |
| BRCA1 ex.20 | | | TGC TCC AC | TAC AG |
| 5382 ins C | | | | |
| Pair 1 for | B1EX5IK1 | identification/ | CTC TTA AGG GCA | TTC CTA CTG TGG TTG |
| BRCA1 ex.5 | | control | GTT GTG AG | CTT CC |
| 300T→G | | | | |
| Pair 2 for | B1EX5IK2 | identification/ | ATG GCT CTT AAG | TGT GGT TGC TTC CAA |
| BRCA1 ex.5 | | control | GGC AGT TG | CCT AG |
| 300T→G | | | | |
| Pair 1 for | B1_4154DELAI1 | identification | CAA AGG CAT CTC | CAA GCC CGT TCC TCT |
| BRCA1 ex.11 | | | AGG AAC ATC | TTC TCA |
| 4153 delA | | | | |
| Pair 2 for | B1_4154DELAI2 | identification | TTG GCT CAG GGT | AAG CCC GTT CCT CTT |
| BRCA1 ex.11 | | | TAC CGA AG | TGT CA |
| 4153 delA | | | | |
| Pair 3 for | B1_4154DELAK1 | control | TTG GCT CAG GGT | GTG CTC CCC AAA AGC |
| BRCA1 ex.11 | | | TAC CGA AG | ATA AAC |
| 4154 delA | | | | |
| Pair 4 for | B1_4154DELAK2 | control | TCC TAG GCC TTT | GTG CTC CCC AAA AGC |
| BRCA1 ex.11 | | | CAC CCA TAC A | ATA AAC |
| 4153 delA | | | | |

TABLE 3
Primer sets for analysis of BRCA1 mutations with multiplex PCR.

| | Primer sets |
|---|---|
| 1 | B1EX5IK1F, B1EX5IK1R, B1_4154DELAI2F, B1_4154DELAI1R, |
| | B1-5382INSCI1F, B1-5382INSCI1R |
| 2 | B1EX5IK2F, B1EX5IK2R, B1_4154DELAK2F, |
| | B1_4154DELAI2R, B1-5382INSCK2F, B1-5382INSCI2R |
| 3 | B1EX5IK1F, B1EX5IK2R B1_4154DELAI2F, B1_4154DELAI2R, |
| | B1-5382INSCI2F, B1-5382INSCI1R |

In order to achieve comparable amounts of amplified PCR products it is in some cases convenient to optimize the applied proportions of primers. Such optimization depends on several factors, e.g. type and activity of polymerase used or the length and composition of the amplified oligonucleotides, and can be achieved based on publicly available laboratory knowledge. Other components for the diagnostic set, besides the primers, include nucleotides, termostable polymerase and buffer for the polymerase reaction, that are necessary elements in the mixture of substances for the PCR reaction.

DNA is isolated from peripheral blood leucocytes by conventional methods, and then used as the matrix for the PCR reaction. Conventional diagnostic tests for mutations in the BRCA1 gene, adjusted for the Polish population, are based on multiplex ASO-PCR (mutations 4153delA and 5382insC) and RFLP (mutation C61G) methods.

The reaction mixture recommended for the aforementioned diagnostic test includes a mixture of primers responsible for
1. amplification of a fragment of exon 5 enclosing the location of the eventual mutation C61G. Additional PCR products are indicators for the quality of the PCR reaction and serve as internal controls. Restriction enzyme AvaII cuts the PCR product of exon 5 into two smaller fragments, whenever mutation C61G is present,
2. amplification of a fragment of exon 11 only in case mutation 4153delA is present in the analyzed material,
3. amplification of a fragment of exon 20 only in case mutation 5382insC is present in the analyzed material,
   where the lengths of the PCR products for exons 5, 11 and 20 are chosen to allow for simple and unequivocal identification using electrophoresis in agarose gel.

Here, the reaction ASO-PCR was carried out in an automatic thermocycler (DNA

ThermalCycler 9600—Perkin Elmer). The mixture of substances for 25 μl volume comprised: 1 μl (50 ng-200 ng) genomic DNA, 2.5 μl reaction buffer (100 mM Tris-HCl, 500 mM KCL, 15 mM MgCl₂, 1 mg/ml gelatin; pH 8.6), 2-14 μM of each primer, 200 μM of each desoxynucleotide (dATP, dCTP, dGTP and dTTP) and 1 U Taq DNA polymerase. For each reaction there are additionally 3 positive controls (control DNA from carriers of the mutations 5382insC, C16G and 4153delA) and 2 negative controls (control DNA from non-carriers and a control with no DNA at all).

Amplification takes place under the following conditions:

a) DNA denaturation at 95° C. during 5 minutes,
b) 10 cycles consisting each of
denaturation at 94° C. during 30 seconds
primer binding at 68-58° C. during 30 seconds*
elongation of complementary DNA at 72° C. during 35 seconds
c) 30 cycles consisting each of
denaturation at 94° C. during 30 seconds
primer binding at 57° C. during 30 seconds
elongation of complementary DNA at 72° C. during 30 seconds
*-for the first 10 cycles the temperature for primer binding is decreased in 1.2° C. for each following cycle (in the first cycle it took 68° C., in the second 66.8° C., in the third 65.6° C., in the fourth 64.4° C., in the fifth 63.2° C., in the sixth 62° C., in the seventh 60.8° C., in the eighth 59.6° C., in the ninth 58.4° C. and in the tenth 57.2° C.).

5 μl of PCR reaction products were mixed with 10 μl Stop buffer (Solution of saccharose stained with bromophenol blue) and subjected to electrophoresis in agarose gel (1.5% agarose SeaKem FMC, 1× bufor TBE, 25 μg/ml ethidium bromide) under 6V/cm for 30 min. The separated products in the gel were visualized with UV illumination.

820 women received neo-adjuvant chemotherapy. Among them, 44 were carriers of one of the above mentioned BRCA1 mutations.

Specific attention was paid to the size of the tumor, prior to and after neoadjuvant chemotherapy and the lymph node status. Pre-treatment tumor size was determined in all patients by a combination of clinical examination and mammography (for some patients ultrasound examinations were also performed). Post-treatment size was determined by pathology report.

Lymph node status was evaluated prior to and after treatment. Pre-treatment lymph node status was evaluated with a combination of clinical exam, ultrasound and fine needle aspiration. After treatment all patients underwent axillary dissection and node status was evaluated by pathology report.

Each study subject was classified, according to response, into complete response “CR” (no evidence of tumor after treatment, either locally or within the axillary nodes), partial response “PR” (residual tumor of size smaller than the original tumor) and no response “BO” (tumor size following treatment equal to, or larger than original tumor size). Patients who experienced a complete response had no residual tumor in the breast tissue upon pathology examination (pathologic complete response).

For each of the 44 BRCA1-positive cases who received neoadjuvant chemotherapy, a matched mutation-negative breast cancer control was selected. The non-carrier control also received neoadjuvant chemotherapy. Carriers and non-carriers were matched on centre, age at diagnosis (within one year) and year of birth (within one year). However, clinical information could only be obtained for 41 of the 44 matched controls. The statistical significance of group differences was assessed using Fisher's Exact Test. Subgroups were defined, based on the results of the ER, PR and ERBB2 immuno-staining and by the category of chemotherapy received (i.e. Taxane containing versus others).

Carrier cases and non-carrier controls are compared in Table 4. Cases and controls were similar with respect to age, tumor size and nodal status. tumors in BRCA1 carriers were more likely to be estrogen-receptor negative, progesterone-negative, ERBB2-negative than tumors in non-carriers (p<0.01 for each). 4 medullary cancers were seen in the BRCA1-positive group, versus none in the non-carriers (p=0.11).

Overall, 35 of the 44 BRCA1 carriers achieved a complete or partial response (80%), compared to 39 of 41 non-carriers (95%; p=0.05) (tables 5 and 6).

A statistically significant difference in the proportions of non-responders in carriers and non-carriers was observed among users of taxane-based regimens. Only 6 of the 15 BRCA1 carriers under docetaxel therapy had a response (complete or partial), compared to 12 of 12 noncarriers (p=0.001). All 29 mutation carriers treated with another treatment regimen (treatments described in table 5) had partial or complete response, compared to 27 of the 29 non-carriers controls. Thus, the inferior response rate to neoadjuvant chemotherapy among BRCA1 carriers was restricted to the subgroup of women given docetaxel. All women who received docetaxel also received doxorubicin (the standard protocol was doxorubicin 50 mg/m2 with docetaxel 75 mg/m2, administered on the same day, for 4 cycles, at 21-day intervals).

BRCA1 carriers were more likely than non-carriers to be negative for estrogen-receptor, progesterone receptor and ERBB2 (table 4). However, the response to docetaxel appeared to be dependent on the BRCA1 status, but not on the receptor status. 27 BRCA1 mutation carriers with ER-negative tumors received treatments that did not contain docetaxel and all 27 achieved a complete or partial response. 12 BRCA1 carriers with ER-negative tumors received docetaxel and doxorubicin. Only 5 experienced a complete or partial response (p=0.0002 for the difference). 4 non-carriers with ER-negative tumors received docetaxel and all 4 responded.

TABLE 4
Characteristics of breast cancer among BRCA1 mutation
carriers and matched non-carriers

| | BRCA1 | |
|---|---|---|
| | mutation | |
| | carriers | Non-carriers |
| | N = 44 | N = 41 |
| Average age | 42.3 years | 42.0 years |

| | | | | |
|---|---|---|---|---|
| Age groups | | | | |
| 20-30 | 3 | 6.8% | 1 | 2.4% |
| 31-40 | 10 | 22.7% | 14 | 34.2% |
| 41-50 | 31 | 70.5% | 26 | 63.4% |
| Tumor histology | | | | |
| Ductal | 24 | 54.5% | 21 | 51.2% |
| Lobular | 1 | 2.3% | 6 | 14.6% |
| Medullary | 4 | 9.1% | 0 | 0 |
| Other | 15 | 34.1% | 14 | 34.2% |
| ER-Status | | | | |
| Positive | 1 | 2.3% | 18 | 43.9% |
| Negative | 40 | 91% | 18 | 43.9% |
| Missing | 3 | 6.8% | 5 | 12.9% |
| PR-Status | | | | |
| Positive | 2 | 4.5% | 11 | 26.8% |
| Negative | 38 | 90.9% | 23 | 56.1% |
| Missing | 4 | 6.8% | 7 | 17.1% |
| ERBB2-Status | | | | |
| Positive | 8 | 18.2% | 11 | 26.8% |
| Negative | 26 | 59.1% | 18 | 43.9% |
| Missing | 10 | 22.7% | 12 | 29.3% |
| Multicentricity | | | | |
| Unicentric | 23 | 52.3% | 21 | 51.2% |
| Multicentric | 6 | 13.6% | 9 | 21.9% |
| Missing | 15 | 34.1% | 11 | 26.9% |
| Tumor size (cm) | | | | |
| <1 cm | 0 | 0% | 0 | 0% |
| 1-2 cm | 4 | 9.1% | 4 | 9.8% |
| 2-5 cm | 30 | 68.2% | 26 | 63.4% |
| >5 cm | 9 | 20.5% | 11 | 26.8% |
| Missing | 1 | 2.2% | 0 | 0 |
| Lymph node status | | | | |
| Negative | 12 | 27.3% | 11 | 26.8% |
| Positive | 32 | 72.3% | 29 | 70.7% |
| Missing | 0 | 0 | 1 | 2.5% |
| Family history on | | | | |
| breast and/or | | | | |
| ovarian cancer | | | | |
| Negative | 4 | 9.1% | 9 | 22% |
| Positive | 37 | 84.1% | 17 | 41.5% |
| Missing | 3 | 6.8% | 15 | 36.5% |

Thus, we observed that women with a BRCA1 mutation who received the spindle poison docetaxel in combination with doxorubicin as neo-adjuvant chemotherapy for breast cancer were significantly less likely to respond to the treatment than women with no mutation. In contrast, BRCA1 carriers who were treated only with alternative DNA-damaging chemotherapies, responded in the same proportion as non-carriers. These observations are consistent with the theory that the expression of the wild-type BRCA protein is necessary for cancer cells to respond to spindle poisons such as docetaxel. BRCA1 may increase cell sensitivity to spindle poisons by signalling a pro-apoptotic pathway in response to spindle damage. In the absence of functional BRCA1, the mitotic spindle checkpoint is not activated and apoptosis is not induced. Supporting that hypothesis, two groups reported that sensitivity to paclitaxel was increased when BRCA1 protein was reconstituted into the tumor cell lines (Lafarge et al. Oncogene 2001; 20:6597-606; Zhoue et al. Oncogene 2003; 22:2396-404) and a third group rendered MCF7 cells insensitive to paclitaxel with a premature inactivation of the spindle checkpoint due to BRCA1 protein downregulation (Chabalier et al. Cell Cycle 2003; 5:1001-7). In contrast, after treatment with DNA-damaging drugs such as anthracyclines, methotrexate and doxorubicin, BRCA1 contributes to DNA repair. The absence of functional BRCA1 protein in BRCA1-mutation carriers should result in poor DNA repair ability, and therefore to an enhanced sensitivity to drugs in this class.

In summary, it was evidenced that breast cancer patients being carriers of BRCA1 germline mutation were significantly less receptive for neoadjuvant cytostatic therapy with taxane-derived drugs than non-carriers, while responsiveness towards DNA-damaging drugs seemed not to be affected by the presence of BRCA 1 mutation.

TABLE 5
Characteristics of BRCA1 mutation carrier breast cancer patients treated with
neoadjuvant chemotherapy

| | | | | | | | Receptors | | | | Response to | | | |
|---|---|---|---|---|---|---|---|---|---|---|---|---|---|---|
| | | | | | | | ER | PgR | HER2 | | chemotherapy | | | Vital |
| 1 | 2 | 3 | 4 | 5 | 6 | 7 | (IHC) | (IHC) | (IHC) | 8 | CR | PR | BO | status |
| 1 | 46 | 2003 | 5382insC | CMF | 10.0 | 1.5 | n.d. | n.d. | n.d. | +/− | | + | | D |
| 2 | 35 | 2002 | C61G | CMF | 4.5 | 3.0 | − | − | +++ | +/+ | | + | | A |
| 3 | 47 | 2003 | 5382insC | CMF | 4.0 | 2.0 | − | − | − | +/+ | | + | | A |
| 4 | 41 | 2000 | 5382insC | CMF | 18. | 5.0 | − | − | n.d. | +/− | | + | | A |
| 5 | 48 | 1998 | 5382insC | CMFP | 4.0 | 2.0 | − | − | − | +/+ | | + | | A |
| 6 | 47 | 1998 | 5382insC | CMF | 3.5 | 1.0 | − | − | ++ | +/− | | + | | A |
| 7 | 47 | 2001 | 5382insC | CMFP | 3.0 | 0.5 | − | − | − | +/− | | + | | A |
| 8 | 48 | 2002 | C61G | CMFP | 2.5 | 0.5 | − | − | − | +/+ | | + | | A |
| 9 | 35 | 2002 | C61G | AC | 6.0 | 2.0 | − | − | − | +/+ | | + | | A |
| 10 | 45 | 2001 | 5382insC | AC | 2.0 | 1.4 | − | − | − | +/− | | + | | A |
| 11 | 46 | 1999 | 5382insC | AC | 3.5 | 1.0 | − | n.d. | n.d. | −/− | | + | | A |
| 12 | 45 | 2001 | 5382insC | AC | 4.0 | 1.3 | − | − | − | +/+ | | + | | A |
| 13 | 44 | 2002 | C61G | AC | 3.4 | 1.0 | − | + | − | −/− | | + | | A |
| 14 | 30 | 2002 | 5382insC | AC | 2.5 | n.d. | − | − | ++ | +/− | + | | | A |
| 15 | 46 | 2002 | 5382insC | FAC | 2.5 | 1.0 | − | − | − | +/− | | + | | A |
| 16 | 40 | 2003 | 5382insC | FAC | 4.5 | 1.5 | − | − | n.d. | +/− | | + | | A |
| 17 | 43 | 2003 | 5382insC | FAC | 5.0 | 3.0 | − | − | − | −/− | | + | | D |
| 18 | 39 | 1996 | C61G | FAC | 4.5 | 2.2 | − | − | ++ | −/− | | + | | A |
| 19 | 38 | 2002 | 5382insC | FAC | 3.0 | 1.0 | − | − | − | −/− | | + | | A |
| 20 | 33 | 2003 | 5382insC | FAC | 2.5 | 1.5 | − | − | ++ | −/− | | + | | A |
| 21 | 44 | 2000 | 5382insC | FAC | 3.0 | 1.5 | − | − | n.d. | −/− | | + | | A |
| 22 | 41 | 1997 | 5382insC | FAC | 15.0 | 7.0 | n.d. | n.d. | n.d. | +/+ | | + | | A |
| 23 | 43 | 2002 | 5382insC | FAC | 3.5 | 2.5 | − | − | − | −/− | | + | | A |
| 24 | 50 | 2003 | C61G | FAC | 6.0 | 3.5 | − | − | ++ | −/− | | + | | A |
| 25 | 43 | 2004 | 5382insC | FAC | 7.0 | n.d. | − | − | − | +/− | + | | | A |
| 26 | 45 | 2004 | 5382insC | FAC | 5.0 | n.d. | − | + | − | +/− | + | | | A |
| 27 | 39 | 2004 | 5382insC | FAC | 5.0 | n.d. | − | − | n.d. | +/− | + | | | A |
| 28 | 31 | 2004 | C61G | AT | 8.0 | 8.0 | n.d. | n.d. | n.d. | +/+ | | | + | D |
| 29 | 30 | 2001 | 5382insC | AT | 4.5 | 5.0 | − | + | n.d. | +/+ | | | + | A |
| 30 | 44 | 2002 | 5382insC | AT | 1.8 | 1.8 | − | − | n.d. | +/+ | | | + | A |
| 31 | 48 | 2002 | C61G | AT | 3.5 | 3.5 | − | − | − | +/+ | | | + | A |
| 32 | 46 | 2002 | 5382insc | AT | 3.5 | 3.5 | ++ | − | − | −/− | | | + | A |
| 33 | 43 | 2003 | 5382insc | AT | 4.0 | 4.0 | − | − | − | −/− | | | + | A |
| 34 | 43 | 2003 | 5382insC | AT | 4.5 | 4.5 | − | − | − | +/+ | | | + | A |
| 35 | 49 | 2003 | 4153delA | AT | 2.5 | 2.5 | − | − | − | +/+ | | | + | D |
| 36 | 43 | 2003 | C61G | AT | 6.5 | 13.0 | − | − | − | +/+ | | | + | D |
| 37 | 46 | 2002 | 5382insC | AT | n.d. | n.d. | − | − | +++ | +/+ | | + | | A |
| 38 | 48 | 2003 | 5382insC | AT | 3.4 | 1.7 | − | − | − | +/+ | | + | | A |
| 39 | 30 | 2001 | C61G | AT | 10.0 | 1.8 | − | − | − | −/− | | + | | A |
| 40 | 45 | 2001 | 4153delA | AT | 2.0 | 1.0 | n.d. | n.d. | n.d. | +/+ | | + | | A |
| 41 | 38 | 2002 | 5382insC | AT | 3.0 | 2.0 | − | − | − | +/+ | | + | | A |
| 42 | 45 | 2004 | 5382insC | AT | 2.5 | n.d. | − | − | +++ | +/+ | | + | | A |
| 43 | 49 | 2000 | 5382insC | CMF | 5.0 | 2.5 | − | − | n.d. | +/+ | | + | | A |
| 44 | 35 | 2001 | 5382insC | CMFP | 2.0 | n.d. | − | − | − | +/+ | | + | | A |

Abbreviations:
CMF - cyclophosphamide (C), methotrexate (M) and fluorouracil (5-FU)
CMFP - cyclophosphamide (C), methotrexate (M), fluorouracil (5-FU) and prednisone (P)
AC - doxorubicin (Adriamycin, A) and cyclophosphamide (C)
FAC - fluorouracil (5-FU), doxorubicin (Adriamycin, A) and cyclophosphamide (C)
AT - doxorubicin (Adriamycin, A) and docetaxel (Taxotere, T)
NA - vinorelbine (Navelbine, N) and doxorubicin (Adriamycin, A)
A—alive
D—deceased
n.d.—no data

TABLE 6
Characteristics of non-carrier breast cancer patients treated with neoadjuvant chemotherapy

| | | | | | | | Receptors | | | | Response to | | | |
|---|---|---|---|---|---|---|---|---|---|---|---|---|---|---|
| | | | | | | | ER | PgR | HER2 | | chemotherapy | | | Vital |
| 1 | 2 | 3 | 4 | 5 | 6 | 7 | (IHC) | (IHC) | (IHC) | 8 | CR | PR | BO | status |
| 1 | 37 | 1997 | — | CMF | 5.0 | 2.0 | + | − | − | +/+ | | + | | D |
| 2 | 37 | 2003 | — | CMF | 6.0 | 1.0 | − | − | − | −/− | | + | | A |
| 3 | 32 | 2003 | — | CMF | 2.5 | 1.5 | − | − | − | +/+ | | + | | A |
| 4 | 47 | 2002 | — | CMF | 2.8 | 0.5 | n.d. | n.d. | n.d. | +/+ | | + | | A |
| 5 | 47 | 1998 | — | CMF | 3.0 | 1.5 | + | − | − | −/− | | + | | A |
| 6 | 50 | 2002 | — | CMF | 4.8 | 2.2 | − | − | ++ | +/+ | | + | | A |
| 7 | 49 | 2003 | — | CMFP | 2.8 | 1.8 | − | − | − | +/+ | | + | | A |
| 8 | 45 | 1998 | — | CMFP | 1.6 | 1.0 | + | − | − | +/+ | | + | | A |
| 9 | 45 | 2002 | — | AC | 4.0 | 1.0 | ++ | + | ++ | +/+ | | + | | D |
| 10 | 40 | 1997 | — | CMFP | 3.8 | n.d. | n.d. | n.d. | n.d. | +/+ | | + | | A |
| 11 | 48 | 2002 | — | AC | 3.0 | 1.5 | ++ | − | − | −/− | | + | | A |
| 12 | 46 | 1999 | — | AC | 4.5 | 1.0 | + | n.d. | n.d. | +/+ | | + | | D |
| 13 | 47 | 2003 | — | AC | 3.5 | 2.5 | ++ | − | − | −/− | | + | | A |
| 14 | 46 | 2002 | — | AC | 1.5 | 0.8 | − | − | n.d. | −/− | | + | | A |
| 15 | 36 | 2002 | — | AC | 2.0 | 2.0 | − | − | +++ | +/+ | | | + | A |
| 16 | 44 | 2002 | — | FAC | 7.0 | 4.0 | + | + | − | −/− | | + | | A |
| 17 | 44 | 2002 | — | FAC | 3.5 | 2.5 | + | ++ | − | +/+ | | + | | A |
| 18 | 45 | 2003 | — | FAC | 3.0 | 1.2 | − | − | +++ | +/+ | | + | | D |
| 19 | 48 | 2003 | — | FAC | 2.0 | 1.2 | − | − | − | +/+ | | + | | A |
| 20 | 40 | 2004 | — | FAC | 8.0 | 0.8 | − | − | n.d. | −/− | | + | | A |
| 21 | 40 | 2000 | — | FAC | 4.5 | 3.0 | ++ | + | n.d. | −/− | | + | | A |
| 22 | 33 | 2003 | — | FAC | 4.0 | n.d. | − | − | − | +/− | + | | | A |
| 23 | 45 | 2003 | — | FAC | 4.0 | 1.5 | ++ | + | − | +/ | | + | | A |
| | | | | | | | | | | n.d. | | | | |
| 24 | 43 | 2003 | — | FAC | 8.0 | 5.0 | − | − | +++ | +/+ | | + | | A |
| 25 | 46 | 2003 | — | FAC | 6.0 | n.d. | − | − | n.d. | −/− | + | | | A |
| 26 | 24 | 2003 | — | FAC | 5.0 | 10.0 | − | − | +++ | +/+ | | | + | A |
| 27 | 43 | 2003 | — | FAC | 7.0 | 5.0 | − | − | +++ | +/+ | | + | | D |
| 28 | 39 | 2003 | — | NA | 4.0 | 1.5 | + | ++ | − | +/+ | | + | | A |
| 29 | 48 | 2001 | — | NA | 3.5 | 1.5 | ++ | +++ | − | +/+ | | + | | A |
| 30 | 34 | 2002 | — | AT | 10.0 | 2.5 | ++ | ++ | − | −/− | | + | | D |
| 31 | 31 | 2002 | — | AT | 5.5 | 2.0 | ++ | + | +++ | +/+ | | + | | D |
| 32 | 32 | 2003 | — | AT | 3.5 | 2.0 | − | − | − | +/+ | | + | | A |
| 33 | 45 | 2001 | — | AT | 4.5 | 2.5 | − | − | n.d. | +/− | | + | | A |
| 34 | 47 | 2003 | — | AT | 6.0 | 3.5 | +++ | + | ++ | +/+ | | + | | A |
| 35 | 44 | 2001 | — | AT | 5.0 | 1.5 | − | n.d. | n.d. | +/+ | | + | | A |
| 36 | 43 | 2003 | — | AT | 6.5 | 4.5 | n.d. | n.d. | n.d. | n.d. | | + | | A |
| 37 | 31 | 2003 | — | AT | 2.5 | 0.7 | + | − | +++ | +/+ | | + | | A |
| 38 | 46 | 2002 | — | AT | 2.8 | 1.4 | n.d. | n.d. | n.d. | −/− | | + | | A |
| 39 | 38 | 2003 | — | AT | 5.0 | 2.0 | + | +++ | − | +/+ | | + | | A |
| 40 | 49 | 2000 | — | AT | 5.8 | 3.8 | − | − | +++ | +/+ | | + | | A |
| 41 | 48 | 2001 | — | AT | 4.5 | 2.0 | n.d. | n.d. | n.d. | +/− | | + | | A |

Abbreviations:
like in foregoing table.

1. A method for selecting a chemotherapeutic agent for providing at least a 50% chance of positive response during treatment of a human subject afflicted with cancer and having a germline alteration in the BRCA1 gene comprising determining whether the human subject has a germline alteration in the BRCA1 gene;
wherein presence of a germline alteration in the BRCA1 gene indicates that a DNA damaging agent at least a 50% chance of positive response if used to treat the human subject, thereby selecting the chemotherapeutic agent.

2. The method of claim 1, wherein the cancer is breast cancer.

3. The method of claim 1, wherein the chance of positive response is at least 60%, or at least 70%, or at least 80%.

4. A method for early detection of reduced clinical response towards cytostatic neoadjuvant chemotherapy in a human cancer patient, which comprises detecting a germline alteration in the sequence of BRCA1 gene in a biological sample from the human patient, wherein the presence of a germline alteration in the sequence of the BRCA1 gene is indicative of reduced clinical response to neoadjuvant chemotherapy with cytostatic drugs in, at least, breast cancer patients.

5. The method of claim 4, wherein alteration is identified by comparison of the structure of the BRCA1 variant of the human subject with the wild type.

6. The method of claim 4, wherein the founder germline mutations of BRCA1 gene being indicative of significantly reduced response to neoadjuvant chemotherapy with cytostatic drugs is identified from a set or panel of BRCA1 founder mutations, which are characteristic for the ethnic population of the patient.

7. The method of claim 1, wherein the founder germline mutations of BRCA1 gene being indicative of significantly reduced response to neoadjuvant chemotherapy with cytostatic drugs is identified from a set or panel of BRCA1 founder mutations, which comprise all known founder mutations of BRCA1 or a sample of the most frequent ones.

8. The method of claim 1, wherein the mode of detection of germline BRCA1 mutations is based on analysis of DNA, RNA or proteins.

9. The method according to claim 8, wherein DNA or RNA testing is performed using any conventional technique of direct mutation detection, such as sequencing, but more preferably any conventional technique of indirect mutation detection, selected among those such as ASA-, ASO-, RFLP-PCR, Taqman RT-PCR or microarrays methods preferably based on common founder mutation panels.

10. The method according to claim 8, wherein the presence of the polypeptide encoded by the BRCA1 gene with germline alteration is detected with the use of antibodies or other substances specific for this polypeptide or its fragment.

11. The method of claim 1, wherein the cytostatic drugs used in pharmacy for cancer chemotherapy are, at least, those derived from taxoid substances, such as paclitaxel (taxol) and docetaxel (taxotere).

12. The method of claim 1, wherein genetic testing is indicated to be performed among all breast cancer patients, for which chemotherapy with cytostatic drugs is intended, but particularly favorable for the case of neoadjuvant therapy for which a quick assignment of the correct chemotherapy is of clinical relevance.

13. The method of identification of genetic markers being predictive of significantly decreased response to neoadjuvant chemotherapy with cytostatic drugs, characterized by comprising the examination of samples containing genomic DNA from patients affected by specific cancer and comparing the frequency of structural change within BRCA1, or regions in linkage disequilibrium, between examined patients and controls from general population, wherein the alteration significantly overrepresented in patients affected by the specific malignancy is then regarded as genetic marker being predictive of significantly decreased response to neoadjuvant chemotherapy with cytostatic drugs in, at least, breast cancer patients.
